(19) United States
(12) Patent Application Publication
(10) Pub. No.: US 2023/0030564 A1
(43) Pub. Date: Feb. 2, 2023

(54) SEPSIS MANAGEMENT

(71) Applicant: ROCHE DIAGNOSTICS OPERATIONS, INC., Indianapolis, IN (US)

(72) Inventors: Andrea Horsch, Luzern (CH); Eberhard Spanuth, Dossenheim (DE)

(21) Appl. No.: 17/772,959

(22) PCT Filed: Oct. 27, 2020
(86) PCT No.: PCT/EP2020/080146

§ 371 Date: Apr. 28, 2022

(30) Foreign Application Priority Data
Oct. 28, 2019 (EP) 19205672.9

(51) Int. Cl. G01N 33/68; G16H 50/20; G16H 50/30

(52) U.S. Cl. CPC G01N 33/6893; G16H 50/20; G16H 50/30; G01N 2333/5753; G01N 2800/26; G01N 2800/50

(57) ABSTRACT

The present invention concerns methods for aiding in the risk assessment of a patient with suspected sepsis. For example, the risk of poor outcome (such as of a complicated clinical course and/or of mortality) can be assessed. The methods of the present invention may comprise the steps of (a) determining the amount of the biomarker Presepsin in a sample from a patient with suspected sepsis who has a known qSOFA (quick Sequential Organ Failure-Assessment) score of 0, 1, 2 or 3, (b) determining the amount of the biomarker Pro-calcitonin (PCT) in a sample from the patient, comparing the amounts determined in steps (b) and (c) to reference amounts, and (d) aiding in the risk assessment of a patient with suspected sepsis. The methods of the present invention may be computer-implemented.

The present invention concerns methods for aiding in the risk assessment of a patient with suspected sepsis. For example, the risk for a poor outcome (such as of a complicated clinical course and/or of mortality) can be assessed. The methods of the present invention may comprise the steps of (a) determining the amount of the biomarker Presepsin in a sample from a patient with suspected sepsis who has a known qSOFA (quick Sequential Organ Failure-Assessment) score of 0, 1, 2 or 3, (b) determining the amount of the biomarker Procalcitonin (PCT) in a sample from the patient, comparing the amounts determined in steps (b) and (c) to reference amounts, and (d) aiding in the risk assessment of a patient with suspected sepsis with regard to clinical outcome. The methods of the present invention may be computer-implemented.

During the initial presentation of a patient with suspected sepsis, for example in the hospital emergency room, it is both challenging and prognostically crucial to rapidly and reliably distinguish between low risk patients with mild sepsis, good prognosis and low in-hospital mortality rate and high risk patients with potentially life-threatening sepsis with need for intensive care and high mortality rates. Early diagnosis and adequate treatment are crucial for the prognosis of patients with sepsis. Patients with sepsis present very differently, which makes clinical assessment difficult. A Dutch study with sepsis patients revealed that 30-50% of the patients, sometimes even those in septic shock, were considered to be ‘non-urgent’ (Eur J Emerg Med 2014; 21(5):330-335. The lack of recognition of the life-threatening clinical picture delays significantly targeted therapy and is associated with increased mortality.

However, in order for sepsis patients to be treated adequately, they first must be identified and the correct diagnosis made. Sepsis is defined in accordance with SEPSIS-3 (Sepsis-3 The Third International Consensus Definitions for Sepsis and Septic Shock. JAMA 2016; 315:801-819) as “a life-threatening organ dysfunction caused by a dysregulated host response to infection” and is recommended to be assessed clinically with the Sequential Organ Failure Assessment (SOFA) score.

The use of the SOFA score is not deemed appropriate for the emergency room due to the complexity of the parameters considered. In order to remedy the situation, the authors of the SEPSIS-3 definition proposed to introduce the quick SOFA (qSOFA) score to predict the likelihood of organ dysfunction in patients with suspected sepsis. The qSOFA score is a three-point score with one point each for tachypnea, hypotension and altered mentation. The “positive” qSOFA score with two or three points indicates risk for a worse clinical outcome. Due to its simplicity, the qSOFA score can be used in the emergency department and in non-intensive areas to identify adult patients with suspected sepsis who are likely to have poor outcomes.

Numerous studies have determined qSOFA to be superior to the previously used SIRS criteria both in terms of specificity and predictive accuracy of the expected mortality rate. In the case of a qSOFA score of ≥2 points, a complicated clinical course or increased mortality is to be expected. However, patients with a qSOFA score of <2 may already be critically ill, if not in need of intensive care, because at least one incipient failure of the circulatory, respiratory or central nervous system is present. Sensitivity of the qSOFA at the time of hospital admission for the correct identification of patients who subsequently die from sepsis during their stay in hospital is only about 50-70% (Lancet Infect Dis 2017; 17:661-670; JAMA 2017; 317:301-308; Chest 2017; 151(3):586-596).

In addition to clinical experience, targeted laboratory examinations to assess the course of the disease and patient's outcome may be helpful. Commonly used inflammatory markers (such as leukocyte count and CRP) are less sensitive or specific. The elevated PCT (Procalcitonin) concentration in the blood indicates the presence of a systemic bacterial infection. Furthermore, in numerous studies, Presepsin (sCD14ST) has been demonstrated to be a useful biomarker for assessing the severity of sepsis (Critical Care 2014; 18:R6 and Critical Care 2013; 17: R244).

The criticism of the new qSOFA score as a screening test, essentially refers to the following points: 1.) Start of therapy may be delayed and therefore cause the prognosis to worsen as the qSOFA score only recognizes patients if they are already in need of intensive care. 2.) High specificity but at the expense of sensitivity. The qSOFA demonstrated significantly lower sensitivity in all previously published prospective studies compared with the SIRS criteria (in favor of a higher specificity) and a negative qSOFA score cannot rule out sepsis. 3.) No biomarkers other than lactate as point-of-care bedside test have been integrated into SEPSIS-3, although various biomarkers for diagnosis, prognosis, and therapy have been used internationally for many years in routine care, on the basis of numerous studies.

There is a clear longstanding need for means and methods for aiding in the quick and reliable assessment of patients with suspected sepsis to adequately predict outcome and for making appropriate therapeutic decision for such patients.

Thus, the technical problem underlying the present invention must be seen as the provision of means and methods for complying with the aforementioned needs. The means and methods shall, at the same time, avoid the drawbacks of the prior art referred to above.

The technical problem is solved by the embodiments characterized in the claims and herein below.

Advantageously, it has been found in the studies underlying the present invention that patients with a suspected sepsis, the combination of quick SOFA scores and the determination of both Procalcitonin and Presepsin in the blood improves the detection of a potentially complicated clinical course and the assessment of the mortality risk. The predictive validity of the qSOFA scores is significantly improved with the addition of both Procalcitonin and Presepsin. In particular, the combined determination of Procalcitonin and Presepsin allows for the identification of risk patients, who—based on the qSOFA alone—would have not been considered to be at risk (such as patients with a qSOFA score of 0 or 1). Further, the combined determination of Procalcitonin and Presepsin allows for the identification of patients whose risk for a poor outcome—based on the qSOFA score alone—would have been overestimated (such as patients with a qSOFA score of 2 or 3). Thanks to the findings of the present invention, it will be possible to make quick and reliable decisions in patients with suspected sepsis who need escalated care to prevent deterioration, such as in patients presenting at an emergency department with suspected sepsis.

Accordingly, the present invention relates to a method for aiding in the risk assessment, such as the prediction of the outcome, of a patient with suspected sepsis, comprising
(a) determining the amount of the biomarker Presepsin in a sample from a patient with suspected sepsis who has a known qSOFA score of 0, 1, 2 or 3,
(b) determining the amount of the biomarker Procalcitonin (PCT) in a sample from the patient,
(c) comparing the amounts determined in steps (b) and (c) to reference amounts, and
(d) aiding in the risk assessment of a patient with suspected sepsis.

Preferably, step (d) is based on the results of the comparison step (c). Accordingly, step (d) preferably comprises aiding in the risk assessment of a patient with suspected sepsis, based on the results of the comparison step.

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and sub-classification of the subject. The method may be carried out manually or assisted by automation. Preferably, step (a), (b), (c) and/or (d) may in total or in part be assisted by automation, e.g., by a suitable robotic and sensory equipment for the determination in step (a) and (b), or a computer-implemented comparison in step (c).

The methods of the present invention shall allow the assessment of a patient with suspected sepsis. The term “assessing” as used herein preferably refers to assessment of the risk of the patient with regard to the clinical course (i.e. need for critical care intervention) and outcome. Thus, the outcome of predicted, i.e. whether the patient is likely, i.e. at risk, of having a poor outcome, or is not likely of having a poor outcome Alternatively, the term “assessing” refers to deciding on suitable therapeutic measures for the patient (e.g. requiring critical care intervention). Thus, the term encompasses the guidance of a patient with suspected sepsis. Accordingly, suggestions for therapeutic measures which are specifically applicable to the patient can be made. By carrying out the methods of the present invention, a patient can be identified who is in need of a certain therapeutic measure, or not.

As will be understood by those skilled in the art, such an assessment is usually not intended to be correct for 100% of the subjects. The expression “assessment” typically requires that an assessment can be made for a statistically significant portion of subjects in a proper and correct manner. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statis-tics for Research, John Wiley & Sons, New York 1983. Preferred confidence intervals are at least 90%, at least 95%, at least 97%, at least 98%, or at least 99%. The p-values are, preferably, 0.1, 0.05, 0.01, 0.005, or 0.0001.

It is to be understood that the actual assessment made in the methods of the present invention may comprise further steps such as the confirmation of the assessment. Thus, the term “assessment”, preferably, is understood as an aid in the assessment. The final assessment may be made by the attending physician, i.e. by the physician who treats the individual.

In a preferred embodiment of the methods of the present invention, the assessment of a patient with suspected sepsis is a risk stratification of the patient, i.e. the assessment of the risk of the tested patient. Accordingly, it is assessed, i.e. predicted, whether the patient is a risk, or not a risk. Accordingly, it is predicted whether the patient is likely to deteriorate (developing multi-organ failure) and having a poor outcome, or is not likely to deteriorate and of having a poor outcome. Said poor outcome shall be associated with sepsis. For example, the risk of mortality, in particular in-hospital mortality, of a septic shock, severe sepsis and/or of a complicated clinical course is predicted.

In a preferred embodiment, the risk of a complicated clinical course is assessed. Thus, the method of the present invention allows for the prediction, whether a patient is at risk, or not at risk, of a complicated clinical course. The risk of complicated clinical course is, preferably, associated with potentially worse outcome and increased in-hospital mortality. Accordingly, the present invention also allows for the prediction of the risk of mortality of the tested patient, particular mortality due to sepsis.

The term “complicated clinical course” is well understood by the skilled person. As used herein, the term is defined as the need for organ support measures required during intensive care unit (ICU) stay, such as administration of intravenous fluids, vasopressors, mechanical ventilation and renal replacement therapy. A patient having complicated clinical course typically requires a longer hospitalization as compared to a patient without complicated clinical course. In some embodiments, a subject who has a clinical complicated course is in need of intensive care measures.

In another preferred embodiment, the risk of a mortality is assessed. Thus, the method of the present invention allows for the prediction, whether a patient is at risk, or not at risk, of mortality due to sepsis. Preferably, said mortality is in-hospital mortality. Further, the method of present invention allows for the prediction of the risk of severe sepsis and/or a septic shock. Thus, it can be assessed whether a patient is at risk of severe sepsis and/or a septic shock, or not.

The terms “assessing the risk” or “predicting the risk” are used interchangeably herein and preferably refer to assessing whether a patient is at risk, or is not at risk. Accordingly, the probability according to which a patient will have complicated clinical course will die and/or will suffer from a septic shock and/or severe sepsis, in particular within a certain predictive window after the method of the present invention has been carried out. Thus, the terms shall mean that the patient to be analyzed by the method of the present invention is allocated either into the group of patients being at risk, or into the group of patients being not at risk (such as of complicated clinical course, mortality (in particular mortality due to sepsis) and/or a septic shock and/or severe sepsis.

In particular, the risk/probability in a certain time window is predicted, e.g. with regard to in-hospital mortality. It is to be understood that the risk that shall be predicted is the short-term risk, i.e. the predictive window is short. In an embodiment the risk of a poor outcome within a period of up to 30 days is predicted. For example the risk of a poor outcome within a period of 3 days to 30 days, or of one week to 30 days, is predicted. In particular, the predictive window is a period of 30 days.

A patient who is at risk has an elevated risk as compared to the average risk, i.e. the patient is a high risk patient with potentially life threatening sepsis. In particular, the risk of the subject is elevated as compared to the average risk of subjects having the same qSOFA score as the subject. A patient who is a risk, i.e. at high risk, is likely to deteriorate and of having a poor outcome and needs escalated care to prevent deterioration, i.e. critical care intervention.

A patient who is not at risk preferably has a reduced risk as compared to the average risk, i.e. the patient is a low risk patient with a good prognosis and low in-hospital mortality risk. In particular, the risk of the subject is reduced as compared to the average risk of subjects having the same qSOFA score as the subject. A patient who is not at risk, i.e. a patient who is at low risk, is likely to have a good outcome.

The “subject” as referred to herein is, preferably, a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the subject is a human subject. The term “patient” and “subject” are used interchangeably herein.

The subject to tested in accordance with the present invention present with suspected sepsis. The term “sepsis” is well-known in the art. As used herein, the term refers a life-threatening organ dysfunction caused by a dysregulated host response to infection. Further, a definition for sepsis can be found in Singer et al. (Sepsis-3 The Third International Consensus Definitions for Sepsis and Septic Shock. JAMA 2016; 315:801-819). In particular, the subject shall be suspected to suffer from a systemic infection. The term “infection” is well understood by the skilled person. As used herein, the term “infection” preferably refers to an invasion of the subject's body tissues by a disease-causing microorganism, its multiplication, and the reaction of subject's tissues to the microorganism. In some embodiments, the infection is a bacterial infection. Thus, the subject shall be suspected to suffer from bacterial sepsis.

A subject with suspected sepsis shows one or more of the following symptoms: faster heart beat (tachycardia), lower systolic blood pressure (hypotension), fever or hypothermia (often with chills), pain, reddened skin, rapid breathing (tachypnoe), difficulty breathing (dyspnoe), inner unrest, dizziness and disorientation. Preferably, the subject has fever, i.e. the subject has a body temperature above the normal range due to an increase in the body's temperature set point. For example, the subject might have a body temperature above 38° C. Alternatively, the subject might have a body temperature above 39° C.

Preferably, the subject with suspected sepsis is a subject who presents at the emergency department. Alternatively, the subject with suspected sepsis is a subject in departments outside of the intensive care unit. Thus, it is envisaged that the subject is not an intensive care patient at the time of the testing (or to be more precise at the time point at which the sample is obtained from the patient.

Preferably, the subject to be tested on accordance with the present invention shall have known qSOFA score (quick Sequential Organ Failure-Assessment score, also referred to as as quickSOFA). The qSOFA score is well known in the art. It was introduced by the Sepsis-3 group in February 2016 as a simplified version of the SOFA Score as an initial way to identify patients at high risk for poor outcome with sepsis. The score is described in by Singer et al. (JAMA. 2016; 315(8):801-810. doi:10.1001/jama.2016.0287) which herewith is incorporated by reference in its entirety with respect its disclosure content. The qSOFA score uses three criteria: assigning one point for low blood pressure (SBP ≤100 mmHg), high respiratory rate (≥22 breaths per min), or altered mentation (Glasgow Coma Scale score <15 points). Thus, the patient's qSOFA score is based on the patient's respiratory rate, the patient's systolic blood pressure, and the presence or absence of an altered mentation.

Typically, a subject has a low blood pressure if the systolic blood pressure is equal to or lower than 100 mmHg. Typically, a subject has an altered mentation, if the subject's Glasgow Coma Scale score is lower than 15 points. Typically, a subject has a low blood pressure if the systolic blood pressure is equal to or lower than 100 mmHg. Typically, a subject has a high respiratory rate, if the subject's respiratory rate is equal to or larger than 22 breaths per min.

In some embodiments, the subject has qSOFA score of 0.

In some embodiments, the subject has qSOFA score of 1.

In some embodiments, the subject has qSOFA score of 2.

In some embodiments, the subject has qSOFA score of 3.

In some embodiments, the subject has qSOFA score of 0 or 1

In some embodiments, the subject has qSOFA score of 2 or 3.

In some embodiments, the subject has qSOFA score of 0, 1, 2 or 3

In preferred embodiments of the methods of the present invention, the subject's qSOFA score is known. Thus, the qSOFA score of the subject has been determined, in particular at or shortly after presentation. In some embodiments, the methods of the present invention encompass the determination of the subject's qSOFA score. In some embodiments, the methods of the present invention do not encompass the determination of the subject's qSOFA score.

The term “sample” refers to a sample of a body fluid, to a sample of separated cells or to a sample from a tissue or an organ. Samples of body fluids can be obtained by well-known techniques and include, samples of blood, plasma, serum, urine, lymphatic fluid, sputum, ascites, or any other bodily secretion or derivative thereof. Preferred body fluid samples are urine, blood, serum or plasma. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

Further, it is envisaged that the sample is a dried blood spot sample. Dried blood spot samples can be obtained by applying drops of blood onto absorbent filter paper. The blood is allowed to thoroughly saturate the paper and is air dried for several hours. The blood may have been drawn by a lancet from the subject to be tested, e.g. from the finger.

In a preferred embodiment, the sample is a blood (i.e. whole blood), serum or plasma sample. Serum is the liquid fraction of whole blood that is obtained after the blood is allowed to clot. For obtaining the serum, the clot is removed by centrifugation and the supernatant is collected. Plasma is the acellular fluid portion of blood. For obtaining a plasma sample, whole blood is collected in anticoagulant-treated tubes (e.g. citrate-treated or EDTA-treated tubes).

Cells are removed from the sample by centrifugation and the supernatant (i.e. the plasma sample) is obtained.

The methods of the present invention are based on the determination of the amounts of two biomarkers, Procalcitonin (abbreviated as PCT) and Presepsin.

Presepsin is also known as “soluble CD14 subtype” or “sCD14-ST” and is derived from sCD14 (soluble CD14), of which at least two forms of higher molecular weight exist (49 kDa and 55 kDa) and of which sCD14-ST is a fragment. Proteolysis of sCD14 leads to formation of Presepsin. The marker is well known in the art and e.g. review in Erenler et al. (Presepsin (sCD14-ST) as a biomarker of sepsis in clinical practice and in emergency department: a mini review. 2015 J Lab Med 39:367-372) which herewith is incorporated by reference. Antibodies which specifically bind to Presepsin, preferably, without binding to sCD14 are available (see e.g. Okamura Y, Yokoi H. Development of a point-of-care assay system for measurement of presepsin (sCD14-ST). Clin Chim Acta. 2011; 412(23-24):2157-61).

Procalcitonin (abbreviated PCT) is a peptide precursor of the hormone calcitonin. Thus, it is the inactive propeptide of calcitonin. It is composed of 116 amino acids and is produced by parafollicular cells (C cells) of the thyroid and by the neuroendocrine cells of the lung and the intestine. PCT is widely reported as a useful biochemical marker to differentiate sepsis from other non-infectious causes of systemic inflammation (Kondo, Y., Umemura, Y., Hayashida, K. et al. J intensive care (2019) 7: 22. https://doi.org/10.1186/s40560-019-0374-4). The amino acid sequence of the marker is well known in the art and is e.g. disclosed in EP2320237B1.

The term “amount” as used herein encompasses the absolute amount of a biomarker as referred to herein (such as PCT and Presepsin), the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts determined from biological read out systems in response to the peptides or intensity signals obtained from specifically bound ligands. It is to be understood that values correlating to the aforementioned amounts or parameters can also be obtained by all standard mathematical operations.

The term “determining” the amount of a biomarker as referred to herein refers to the quantification of the biomarker, e.g. to determining the level of the biomarker in the sample, employing appropriate methods of detection described elsewhere herein.

In an embodiment, the amount of a biomarker is determined by contacting the sample with an agent that specifically binds to the biomarker, thereby forming a complex between the agent and said biomarker, detecting the amount of complex formed, and thereby determining the amount of said biomarker.

The biomarker as referred to herein can be detected using methods generally known in the art. Methods of detection generally encompass methods to quantify the amount of a biomarker in the sample (quantitative method). It is generally known to the skilled artisan which of the following methods are suitable for qualitative and/or for quantitative detection of a biomarker. Samples can be conveniently assayed for, e.g., proteins using Westerns and immunoassays, like ELISAs, RIAs, fluorescence- and luminescence-based immunoassays, which are commercially available. Further suitable methods to detect biomarker include determining a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR-analyzers, or chromatography devices. Further, methods include microtiter plate ELISA-based methods, fully-automated or robotic immunoassays (available for example on Roche Elecsys™ analyzers), and latex agglutination assays (available for example on Roche-Hitachi™ analyzers). In addition, LC-MS (Liquid Chromatography-Mass Spectrometry) can be used for the detection and quantification of peptides and proteins in biological matrices.

For the detection of biomarker proteins as referred to herein a wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker. Sandwich assays are among the most useful immunoassays.

Methods employing electrochemiluminescent labels are well-known. Such methods make use of the ability of special metal complexes to achieve, by means of oxidation, an excited state from which they decay to ground state, emitting electrochemiluminescence. For review see Richter, M. M., Chem. Rev. 104 (2004) 3003-3036.

In an embodiment, the detection antibody (or an antigen-binding fragment thereof) to be used for determining the amount of a biomarker is ruthenylated or iridinylated. Accordingly, the antibody (or an antigen-binding fragment thereof) shall comprise a ruthenium label. In an embodiment, said ruthenium label is a bipyridine-ruthenium(II) complex. Or the antibody (or an antigen-binding fragment thereof) shall comprise an iridium label. In an embodiment, said iridium label is a complex as disclosed in WO 2012/107419.

Determining the amount of a peptide or polypeptide (such as PCT or Presepsin) may, preferably, comprise the steps of (a) contacting the peptide or polypeptide with an agent that specifically binds said polypeptide (b) (optionally) removing non-bound agent, (c) determining the amount of bound binding agent, i.e. the complex of the agent formed in step (a). According to a preferred embodiment, said steps of contacting, optionally removing and determining may be performed by an analyzer unit. According to some embodiments, said steps may be performed by a single analyzer unit of said system or by more than one analyzer unit in operable communication with each other. For example, according to a specific embodiment, said system disclosed herein may include a first analyzer unit for performing said steps of contacting and optionally removing and a second analyzer unit, operably connected to said first analyzer unit by a transport unit (for example, a robotic arm), which performs said step of determining.

The agent which specifically binds the biomarker (herein also referred to as “binding agent”) may be coupled covalently or non-covalently to a label allowing detection and measurement of the bound agent. Labeling may be done by direct or indirect methods. Direct labeling involves coupling of the label directly (covalently or non-covalently) to the binding agent. Indirect labeling involves binding (covalently or non-covalently) of a secondary binding agent to the first binding agent. The secondary binding agent should specifically bind to the first binding agent. Said secondary binding agent may be coupled with a suitable label and/or be the target (receptor) of tertiary binding agent binding to the secondary binding agent.

Suitable secondary and higher order binding agents may include antibodies, secondary antibodies, and well-known binding-systems such as the streptavidin-biotin system (Vector Laboratories, Inc.). The binding agent or substrate may also be “tagged” with one or more tags as known in the art. Such tags may then be targets for higher order binding agents. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium complexes, iridium complexes, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, avail-able as ready-made stock solution from Roche Diagnostics), CDP-Star™ (Amersham Bio-sciences), ECF™ (Amersham Biosciences). A suitable enzyme-substrate combination may result in a colored reaction product, fluorescence or chemoluminescence, which can be determined according to methods known in the art (e.g. using a light-sensitive film or a suit-able camera system). As for determining the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. A radioactive label can be detected by any method known and appropriate, e.g. a light-sensitive film or a phosphor imager.

The amount of a polypeptide may be, also preferably, determined as follows: (a) contacting a solid support comprising a binding agent for the polypeptide as described elsewhere herein with a sample comprising the peptide or polypeptide and (b) determining the amount of peptide or polypeptide which is bound to the support. Materials for manufacturing supports are well-known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc.

In yet an aspect the sample is removed from the complex formed between the binding agent and one marker prior to the measurement of the amount of formed complex. Accordingly, in an aspect, the binding agent may be immobilized on a solid support. In yet an aspect, the sample can be removed from the formed complex on the solid support by applying a washing solution.

“Sandwich assays” are among the most useful and commonly used assays encompassing a number of variations of the sandwich assay technique. Briefly, in a typical assay, an unlabeled (capture) binding agent is immobilized or can be immobilized on a solid substrate, and the sample to be tested is brought into contact with the capture binding agent. After a suitable period of incubation, for a period of time sufficient to allow formation of a binding agent-biomarker complex, a second (detection) binding agent labeled with a reporter molecule capable of producing a detectable signal is then added and incubated, allowing time sufficient for the formation of another complex of binding agent-biomarker-labeled binding agent. Optionally, any unreacted material may be washed away. The presence of the biomarker is determined by observation of a signal produced by the reporter molecule bound to the detection binding agent. The results may either be qualitative, by simple observation of a visible signal, or may be quantitated by comparison with a control sample containing known amounts of biomarker.

The incubation steps of a typical sandwich assays can be varied as required and appropriate. Such variations include for example simultaneous incubations, in which two or more of binding agent and biomarker are co-incubated. For example, both, the sample to be analyzed and a labeled binding agent are added simultaneously to an immobilized capture binding agent. It is also possible to first incubate the sample to be analyzed and a labeled binding agent and to thereafter add an antibody bound to a solid phase or capable of binding to a solid phase.

The formed complex between a specific binding agent and the biomarker shall be proportional to the amount of the biomarker present in the sample. It will be understood that the specificity and/or sensitivity of the binding agent to be applied defines the degree of proportion of at least one marker comprised in the sample which is capable of being specifically bound. Further details on how the measurement can be carried out are also found elsewhere herein. The amount of formed complex shall be transformed into an amount of the biomarker reflecting the amount indeed present in the sample.

In some embodiments, the amount of Presepsin is determined with the Pathfast Presep sin assay from LSI Medience Corporation (13-4, Uchikanda 1-chome, Chiyoda-ku, Tokyo, Japan, Product No. PF 1201-K).

In some embodiments, the amount of PCT is determined with the Elecsys® BRAHMS PCT assay from Roche Diagnostics GmbH, 68305 Mannheim, Germany.

The terms “binding agent”, “specific binding agent”, “analyte-specific binding agent”, “detection agent” and “agent that specifically binds to a biomarker” are used interchangeably herein. Preferably, it relates to an agent that comprises a binding moiety which specifically binds the corresponding biomarker. Examples of “binding agents” or “agents” are a nucleic acid probe, nucleic acid primer, DNA molecule, RNA molecule, aptamer, antibody, antibody fragment, peptide, peptide nucleic acid (PNA) or chemical compound. A preferred agent is an antibody, or antigen-binding fragment thereof, which specifically binds to the biomarker to be determined. The term “antibody” herein is used in the broadest sense and encompasses various antibody structures, including but not limited to monoclonal antibodies, polyclonal antibodies, multispecific antibodies (e.g., bispecific antibodies), and antibody fragments as long as they exhibit the desired antigen-binding activity (i.e. antigen-binding fragments thereof). Preferably, the antibody is a polyclonal antibody. More preferably, the antibody is a monoclonal antibody.

The term “specific binding” or “specifically bind” refers to a binding reaction wherein binding pair molecules exhibit a binding to each other under conditions where they do not significantly bind to other molecules. The term “specific binding” or “specifically binds”, when referring to a protein or peptide as biomarker, refers to a binding reaction wherein a binding agent binds to the corresponding biomarker with an affinity of at least 10⁻⁷ M. The term “specific binding” or “specifically binds” preferably refers to an affinity of at least 10⁻⁷ M or even more preferred of at least 10⁻⁹ M for its target molecule. The term “specific” or “specifically” is used to indicate that other molecules present in the sample do not significantly bind to the binding agent specific for the target molecule.

The term “comparing” as used herein refers to comparing the amount of the biomarker in the sample from the subject with the reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer-assisted. Thus, the comparison may be carried out by a computing device. The value of the determined or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provides the desired assessment in a suitable output format.

In accordance with the present invention the amount of the biomarker PCT and the biomarker Presepsin shall be compared to a reference. Typically, the amount of the biomarker PCT is compared to a reference for PCT, and the amount of the biomarker Presepsin is compared to a reference for Presepsin.

The reference is preferably a reference amount. The term “reference amount” as used herein refers to an amount which allows for allocation of a subject into either (i) the group of subjects being at risk or (ii) the group of being not at risk (e.g. of a complicated clinical course). A suitable reference amount may be determined from a reference sample to be analyzed together, i.e. simultaneously or subsequently, with the test sample.

Reference amounts can, in principle, be calculated for a cohort of subjects as specified above based on the average or mean values for a given biomarker by applying standard methods of statistics. In particular, accuracy of a test such as a method aiming to diagnose an event, or not, is best described by its receiver-operating characteristics (ROC) (see especially Zweig 1993, Clin. Chem. 39:561-577). The ROC graph is a plot of all of the sensitivity versus specificity pairs resulting from continuously varying the decision threshold over the entire range of data observed. The clinical performance of a prognostic method depends on its accuracy, i.e. its ability to correctly allocate subjects to a certain prognosis. The ROC plot indicates the overlap between the two distributions by plotting the sensitivity versus 1- specificity for the complete range of thresholds suitable for making a distinction. On the y-axis is sensitivity, or the true-positive fraction, which is defined as the ratio of number of true-positive test results to the product of number of true-positive and number of false-negative test results. This has also been referred to as positivity in the presence of a disease or condition. It is calculated solely from the affected subgroup. On the x-axis is the false-positive fraction, or 1—specificity, which is defined as the ratio of number of false-positive results to the product of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/1—specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis for a given event with a proper balance of sensitivity and specificity, respectively. Accordingly, the reference to be used for the aforementioned method of the present invention, i.e. a threshold which allows to differentiating between subjects who are at risk or those who are not at risk among a cohort of subjects with suspected sepsis can be generated, preferably, by establishing a ROC for said cohort as described above and deriving a threshold amount therefrom. Dependent on a desired sensitivity and specificity for a diagnostic method, the ROC plot allows deriving a suitable threshold. It will be understood that an optimal sensitivity is desired for excluding a subject who is at risk, (i.e. a rule out) whereas an optimal specificity is envisaged for a subject to be assessed as being at risk (i.e. a rule in).

In certain embodiments, the term “reference amount” as used herein refers to a predetermined value. Said predetermined value shall allow for differentiating between a subject who is at risk and a subject who is not at risk (such as of a complicated clinical course).

For example, the reference amount for Presepsin is within the range from about 500 pg/mL to about 1500 pg/mL. Preferably, the reference amount for Presepsin is within the range from about 750 pg/mL to about 1250 pg/mL, more preferably within the range from about 850 pg/mL to about 1150 pg/mL, even more preferably within the range from about 950 pg/mL to about 1050 pg/mL. Most preferably, the reference amount is about 1000 pg/mL.

Preferably, the reference amount for PCT is within the range from about 1.5 ng/mL to about 2.5 ng/mL, more preferably within the range from about 1.75 ng/mL to about 2.25 ng/mL, even more preferably within the range from about 1.9 ng/mL to about 2.1 ng/mL. Most preferably, the reference amount for PCT is about 2 ng/mL.

In the following preferred diagnostic algorithms are summarized.

Preferably, an amount of Presepsin in the sample from the subject which is larger than the reference amount for Presepsin and/or an amount of PCT in the sample from the subject which is larger than the reference amount for PCT is indicative for a subject who is at risk (in particular of a complicated clinical course). Thus, a subject is at risk, if i) the amount of Presepsin is increased, if ii) the amount of PCT is increased, or if iii) both the amounts of Presepsin and PCT are increased as compared to the corresponding reference.

Additionally or alternatively, an amount of Presepsin in the sample from the subject which is lower than the reference amount for Presepsin and an amount of PCT in the sample from the subject which is lower than the reference amount for PCT is indicative for a subject who is at not at risk (in particular of a complicated clinical course). Thus, the subject is not at risk if both the amounts of Presepsin and PCT are decreased as compared to the corresponding reference.

The rule in of the risk is particularly advantageous for subjects with a “negative” qSOFA score, such as for subjects with a qSOFA score of 0 or 1. In a preferred embodiment, the subject thus has a (known) qSOFA score of 0 or 1, wherein an amount of Presepsin in the sample from the subject which is larger than the reference amount for Presepsin and/or an amount of PCT in the sample from the subject which is larger than the reference amount for PCT is indicative for a subject who is at risk.

The rule out of the risk is particularly advantageous for subjects with a “positive” qSOFA score, such as for subjects with a qSOFA score of 2 or 3. In a preferred embodiment, the subject thus has a (known) qSOFA score of 2 or 3, an amount of Presepsin in the sample from the subject which is lower than the reference amount for Presepsin and an amount of PCT in the sample from the subject which is which is lower than the reference amount for PCT is indicative for a subject who is at not at risk (in particular of a complicated clinical course).

In a preferred embodiment of the methods of the present invention, the methods further comprise recommending or initiating a suitable therapeutic measure. Preferably, said suitable therapeutic measure is selected from the medical guidelines or recommendations for management of sepsis such as International Guidelines for Management of Sepsis and Septic Shock (Intensive Care Med, 2017). In particular, the therapeutic measure may be treatment of sepsis or further diagnostic investigation or other aspects of care deemed necessary by the practitioners.

In an embodiment, the therapeutic measure to be recommended or initiated if a patient has been assessed to be at risk is selected from
administration of empiric broad spectrum therapy with at least one or more (i.e. combination therapy) antimicrobial agents such as a cephalosporine, a beta-lactam/beta-lactamase inhibitor (e.g. piperacillin) and a carbapenem, preferably depending on the organisms that are considered likely pathogens and antibiotic susceptibilities
fluid resuscitation
administration of one or more vasopressors, such as administration of norepinephrine
administration of one or more corticosteroids, such as administration of hydrocortisone
renal replacement therapy, such as dialysis, and/or
mechanical ventilation.

The definitions and explanations given herein above preferably apply mutatis mutandis to the following methods of the present invention.

As set forth above, the above method of the present invention may encompass obtaining or the determination of the patients's qSOFA score. Accordingly, the present invention relates to a method for aiding in the risk assessment of a patient with suspected sepsis, comprising
(a) obtaining or determining the patient's qSOFA (quick Sequential Organ Failure-Assessment) score,
(b) determining the amount of the biomarker Presepsin in a sample from the patient with suspected sepsis
(c) determining the amount of the biomarker Procalcitonin (PCT) in a sample from the patient,
(d) comparing the amounts determined in steps (b) and (c) to reference amounts, and
(e) aiding in the risk assessment of the patient.

Preferably, step (e) is based on the qSOFA score obtained or determined in step (a) and on the results of the comparison step (c). Accordingly, step (e) preferably comprises aiding in the risk assessment of a patient with suspected sepsis based on both the qSOFA score and on the results of the comparison step.

The determination of the patient's qSOFA score preferably encompasses the assessment of the patient's respiratory rate, the patient's systolic blood pressure, and the assessment of presence or absence of an altered mentation in the patient, and the calculation of the qSOFA score based on the aforementioned assessment.

Obtaining the patient's qSOFA score preferably means receiving a value for the patient's qSOFA score. Thus, obtaining the patient's qSOFA score does not encompass any active diagnostic steps.

The methods of the present invention may be also carried out as computer-implemented inventions.

Accordingly, the present invention relates to a computer-implemented method for the assessment of a patient with suspected sepsis, comprising
(a) receiving at a processing unit
   (a1) a value for the amount of the biomarker Presepsin in a sample from a patient with suspected sepsis who has a known qSOFA (quick Sequential Organ Failure-Assessment) score of 0, 1, 2 or 3 and
   (a2) a value for the amount of the biomarker Procalcitonin in a sample from the patient,
(b) processing the values received in step (a) with the processing unit, wherein said processing comprises
   (b1) retrieving from a memory one or more threshold values for the amount of the biomarker Presepsin, and one or more threshold values for the amount of the biomarker Procalcitonin,
   (b2) comparing the values received in step (a) with the respective threshold values retrieved in step (b1), and
(c) providing an assessment of the patient via an output device, wherein said assessment is based on the results of step b).

Alternatively, the present invention relates to computer-implemented method for the assessment of a patient with suspected sepsis, comprising
(a) receiving at a processing unit
   (a1) a value for the patient's qSOFA (quick Sequential Organ Failure-Assessment) score,
   (a2) a value for the amount of the biomarker Presepsin in a sample from the patient, and
   (a3) a value for the amount of the biomarker Procalcitonin in a sample from the patient,
(b) processing the values received in step (a) with the processing unit, wherein said processing comprises
   (b1) retrieving from a memory a threshold value for the qSOFA score, one or more threshold values for the amount of the biomarker Presepsin, and one or more threshold values for the amount of the biomarker Procalcitonin,
   (b2) comparing the values received in step (a) with the respective threshold values retrieved in step (b 1), and
(c) providing an assessment of the patient via an output device, wherein said assessment is based on the results of step b).

In an embodiment of the methods of the present invention, information on the assessment (according to the last step of the methods of the present invention) is provided via a display, configured for presenting the assessment. Accordingly, information may be provided whether the subject with suspected sepsis is at risk for developing a complicated clinical course, or is not at risk as described elsewhere herein. Further, recommendations for suitable therapeutic can be displayed. As described elsewhere herein, various alternative therapeutic measures may be recommended. In this case, the treatment option or treatment option(s) may be shown in the display

In an embodiment of the methods of the present invention, the methods may comprise the further step of transferring the information on the assessment of the methods of the present invention to the subject's electronic medical records.

Alternatively, the assessment made in the last step of the methods of the present invention can be printed by a printer. The print-out shall contain information on whether the patient is at risk, or not at risk and/or a recommendation of a suitable therapeutic measure.

In an embodiment of the methods of the present invention, a subject who has been identified as being at risk, or not at risk of a complicated clinical course is treated based on the risk. Thus, a suitable therapeutic measure is initiated, depending on whether the subject is at risk or is not at risk.

The present invention thus further relates to a method of treating a patient with suspected sepsis, the method comprising, carrying out any of the methods of the present invention for assessing a patient with suspected sepsis, thereby identifying an individual who is at risk or not at risk, and initiating a suitable therapeutic method.

The present invention further relates to computer program including computer-executable instructions for performing the steps of the method according to the present invention of assessing a subject with suspected sepsis, when the program is executed on a computer or computer network. Typically, the computer program specifically may contain computer-executable instructions for performing the steps of the method as disclosed herein. Specifically, the computer program may be stored on a computer-readable data carrier.

The present invention further relates to computer program product with program code means stored on a machine-readable carrier, in order to perform the method according to present invention, when the program is executed on a computer or computer network, such as one or more of the above-mentioned steps discussed in the context of the computer program. As used herein, a computer program product refers to the program as a tradable product. The product may generally exist in an arbitrary format, such as in a paper format, or on a computer-readable data carrier. Specifically, the computer program product may be distributed over a data network.

The present invention further relates to a computer or computer network comprising at least one processing unit, wherein the processing unit is adapted to perform all steps of the computer-implemented method according to the present invention.

Yet, the present invention also contemplates:
A computer or computer network comprising at least one processing unit, wherein said processing unit is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising glucose data measurements obtained from the individual as specified herein above, and a data stream signal, typically encrypted, comprising an information providing an aid in the assessment of guidance obtained by the method of the invention.

The present invention further relates to a device for assessing a subject with suspected sepsis, said device comprising a processing unit, and a computer program including computer-executable instructions, wherein said instructions, when executed by the processing unit, causes the processing unit to perform the computer-implemented method according to the present invention, i.e. to perform the steps of said method. In an embodiment, steps a) to d) of the method of the present invention are performed by the processing unit. The device may further comprise a user interface and a display, wherein the processing unit is coupled to the user interface and the display. Typically, the device provides as output the assessment of the patient and/or a recommendation for a suitable therapeutic measure. In an embodiment, the output is provided on the display.

The present invention further relates to i) the use of PCT and Presepsin as biomarkers, or to ii) the use of at least one detection agent which specifically binds to PCT and of at least one detection agent which specifically binds to Presepsin, for assessing the risk of a patient with suspected sepsis, wherein said patient has a known qSOFA score of 0, 1, 2 or 3. Preferably, said use in an in vivo use.

Preferred detection agents are disclosed elsewhere herein (such as antibodies, or antigen binding fragments thereof).

### List of Embodiments

In the following, preferred embodiments are summarized. The definitions and explanations given herein above preferably apply mutatis mutandis to the following embodiments.
1. A method for aiding in the risk assessment of a patient with suspected sepsis, comprising
   (a) determining the amount of the biomarker Presepsin in a sample from a patient with suspected sepsis who has a known qSOFA (quick Sequential Organ Failure-Assessment) score of 0, 1, 2 or 3
   (b) determining the amount of the biomarker Procalcitonin (PCT) in a sample from the patient,
   (c) comparing the amounts determined in steps (b) and (c) to reference amounts, and
   (d) aiding in the risk assessment of a patient with suspected sepsis.
2. The method of embodiment 1, wherein the patient is a human patient.
3. The method of embodiments 1 and 2, wherein the sample is a blood, serum or plasma sample.
4. The method of embodiments 1 to 3, wherein the patient is a patient who presents with suspected sepsis.
5. The method of any one of embodiments 1 to 4, wherein the risk of mortality, such as in-hospital mortality, a complicated clinical course, severe sepsis and/or septic shock is assessed.
6. The method of embodiment 5, wherein the complicated clinical course is defined as the need for organ support measures required during intensive care unit (ICU) stay, such as administration of intravenous fluids, vasopressors, mechanical ventilation or renal replacement therapy.
7. The method of any one of embodiments 1 to 6, wherein the subject has a known qSOFA (quick Sequential Organ Failure-Assessment) score of 0 or 1.
8. The method of embodiment 7, wherein an amount of Presepsin in the sample from the subject which is larger than the reference amount for Presepsin and/or an amount of PCT in the sample from the subject which is larger than the reference amount for PCT is indicative for a subject who is at risk.
9. The method of embodiment 7, wherein an amount of Presepsin in the sample from the to subject which is lower than the reference amount for Presepsin and an amount of PCT in the sample from the subject which is which is lower than the reference amount for PCT is indicative for a subject who is at not at risk.
10. The method of any one of embodiments 1 to 6, wherein the subject has a known qSOFA (quick Sequential Organ Failure-Assessment) score of 2 or 3.
11. The method of embodiment 10, wherein an amount of Presepsin in the sample from the subject which is larger than the reference amount for Presepsin and/or an amount of PCT in the sample from the subject which is larger than the reference amount for PCT is indicative for a subject who is at risk.
12. The method of embodiment 10, wherein an amount of Presepsin in the sample from the subject which is lower than the reference amount for Presepsin and an amount of PCT in the sample from the subject which is which is lower than the reference amount for PCT is indicative for a subject who is at not at risk.
13. The method of any one of embodiments 1 to 12, further comprising recommending or initiating a suitable therapeutic measure.
14. The method of embodiment 13, wherein the therapeutic measure is selected from recommended guidelines for management of sepsis, if the subject has been assessed to be at risk.
15. The method of embodiment 13, wherein the therapeutic measure may be treatment of infection or further investigation or other aspects of care deemed necessary by the practitioners, if the subject has been assessed to be not at risk.
16. The method of any one of embodiments 1 to 15, wherein the reference amount for Presepsin is within the range from about 500 pg/mL to about 1500 pg/mL, or about 750 pg/mL to about 1250 pg/mL, e.g. wherein the reference amount for Presepsin is about 1000 pg/mL, and/or wherein the reference amount for PCT is within the range from about 1.5 ng/mL to about 2.5 ng/mL, e.g. wherein the reference amount for PCT is about 2 ng/mL.
17. A method for aiding in the risk assessment of a patient with suspected sepsis, comprising
   (a) obtaining the patient's qSOFA (quick Sequential Organ Failure-Assessment) score,
   (b) determining the amount of the biomarker Presepsin in a sample from the patient with suspected sepsis
   (c) determining the amount of the biomarker Procalcitonin (PCT) in a sample from the patient,
   (d) comparing the amounts determined in steps (b) and (c) to reference amounts, and
   (e) aiding in the risk assessment of the patient.
18. The method of embodiment 17, wherein the patient's qSOFA score is based on the patient's respiratory rate (>22/min), the patient's systolic blood pressure (<100 mmHg), and the presence or absence of an altered mentation (GCS <15).
19. A computer-implemented method for the assessment of a patient with suspected sepsis, comprising
   (a) receiving at a processing unit
      (a1) a value for the amount of the biomarker Presepsin in a sample from a patient with suspected sepsis who has a known qSOFA (quick Sequential Organ Failure-Assessment) score of 0, 1, 2 or 3 and
      (a2) a value for the amount of the biomarker Procalcitonin in a sample from the patient,
   (b) processing the values received in step (a) with the processing unit, wherein said processing comprises
      (b1) retrieving from a memory one or more threshold values for the amount of the biomarker Presepsin, and one or more threshold values for the amount of the biomarker Procalcitonin,
      (b2) comparing the values received in step (a) with the respective threshold values retrieved in step (b 1), and
   (c) providing an assessment of the patient via an output device, wherein said assessment is based on the results of step b).
20. A computer-implemented method for the assessment of a patient with suspected sepsis, comprising
   (a) receiving at a processing unit
      (a1) a value for the patient's qSOFA (quick Sequential Organ Failure-Assessment) score,
      (a2) a value for the amount of the biomarker Presepsin in a sample from the patient, and
      (a3) a value for the amount of the biomarker Procalcitonin in a sample from the patient,
   (b) processing the values received in step (a) with the processing unit, wherein said processing comprises
      (b1) retrieving from a memory a threshold value for the qSOFA score, one or more threshold values for the amount of the biomarker Presepsin, and one or more threshold values for the amount of the biomarker Procalcitonin,
      (b2) comparing the values received in step (a) with the respective threshold values retrieved in step (b 1), and
   (c) providing an assessment of the patient via an output device, wherein said assessment is based on the results of step b).
21. The method of embodiment 19 or 20, wherein the output device is a display, configured for presenting the assessment.
22. The method of any one of embodiments 19 to 22, wherein the value for the amount of the biomarker Presepsin and/or Procalcitonin is the value for the amount of the biomarker in a blood, serum or plasma sample.
23. The method of any one of embodiments 1 to 22, wherein the patient with suspected sepsis shows one or more of the following symptoms: tachycardia, hypotension, fever or hypothermia, pain, reddened skin, tachypnoe, dyspnoe, inner unrest, dizziness and disorientation, and/or wherein the patient's qSOFA score is based on the patient's respiratory rate (>22/min), the patient's systolic blood pressure (<100 mmHg), and the presence or ab-sence of an altered mentation (GCS <15).
24. A device for the assessment of a patient with suspected sepsis, said device comprising a processing unit and a computer program including computer-executable instructions, wherein said instructions, when executed by the processing unit, causes the processing unit to perform the computer-implemented method of any one of embodiments 19 to 23.
25. The device of embodiment 24, wherein the processing is coupled to a user interface and a display.
26. A method for the assessment of a patient with suspected sepsis, comprising
   (a) obtaining a value for the patient's qSOFA (quick Sequential Organ Failure-Assessment) score,
   (b) determining the amount of the biomarker Presepsin in a sample from the patient,
   (c) determining the amount of the biomarker Procalcitonin in a sample from the patient,
   (d) comparing the value obtained in step a) to a reference value, comparing the amounts determined in steps (b) and (c) to reference amounts, and
   (e) assessing the patient based on the results of step d).
27. A method for improving the accuracy of the qSOFA score, comprising
   (a) determining the amount of the biomarker Presepsin in a sample from a patient having a known qSOFA score of 0, 1, 2 or 3,
   (b) determining the amount of the biomarker Procalcitonin in a sample from the patient,
   (c) comparing the amounts determined in steps (a) and (b) to reference amounts, and
   (d) improving the accuracy of the qSOFA score based on the results of step (c).
28. Use of PCT and Presepsin as biomarker, or to ii) the use of at least one detection agent which specifically binds to PCT and of at least one detection agent which specifically binds to Presepsin, for assessing the risk of a patient with suspected sepsis, wherein said patient has a known qSOFA score of 0, 1, 2 or 3.

All references cited in this specification are herewith incorporated by reference with respect to their entire disclosure content and the disclosure content specifically mentioned in this specification.

In the Figures:

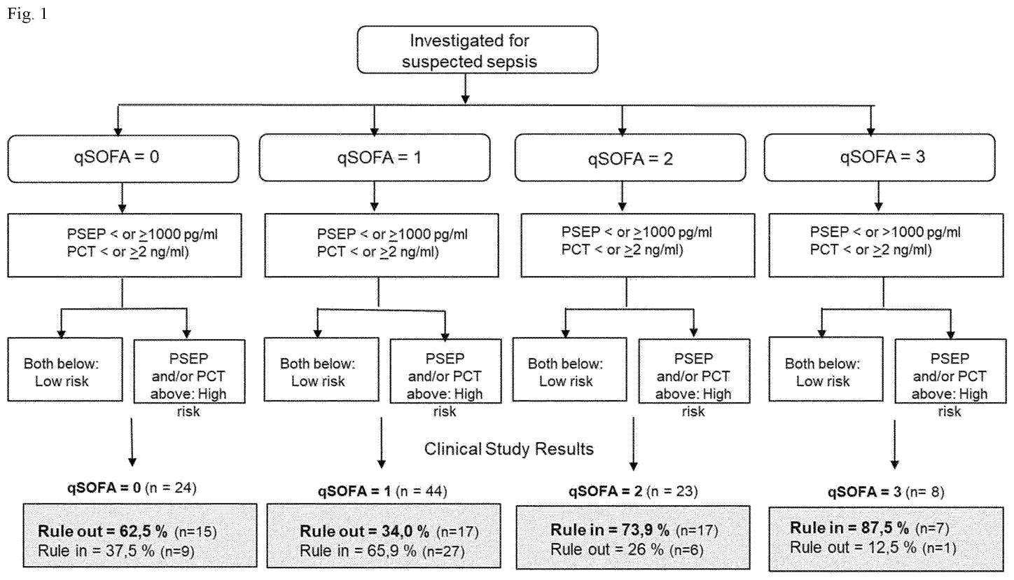

FIG. 1 Combined assessment of Presepsin (PSEP) and Procalcitonin (PCT) in addition to the qSOFA score improves the prediction of a complicated clinical course (severe sepsis, septic shock and mortality) in patients with early sepsis admitted to the emergency department

### EXAMPLES

The invention will be merely illustrated by the following Examples. The said Examples shall, whatsoever, not be construed in a manner limiting the scope of the invention.

### Example 1: Assessment of Presepsin (PSEP), Procalcitonin (PCT) and the Quick SOFA Score in Patients with Suspected Sepsis

Presepsin (soluble sCD14 subtype, sCD14-ST) is a circulating molecule fragment derived from sCD14 and serves as mediator of lipopolysaccharid (LPS) response against infectious agents. Presepsin has been shown to be beneficial as sepsis marker.

PCT is a member of the calcitonin (CT) superfamily of peptides. Due to PCT's variance between microbial infections and healthy individuals, it has become a marker to improve identification of systemic bacterial infection. Measurement of Procalcitonin can be used as a marker of severe sepsis caused by bacteria and correlates with the degree of sepsis.

The Sequential Organ Failure Assessment (SOFA) score was documented as well as respiratory rate, systolic blood pressure (RRsyst) and altered mentation (GCS score) enabling the calculation of the Quick SOFA (qSOFA) score retrospectively.

In 99 patients with suspected sepsis admitted to the emergency department (ED) Presepsin (PSEP), Procalcitonin (PCT) and the SOFA score were determined upon admission. Additional measured parameters were CRP, creatinine and lactate. The Sequential Organ Failure Assessment (SOFA) score was documented as well as respiratory rate, systolic blood pressure (RRsyst) and altered mentation (GCS score) to calculate the Quick SOFA (qSOFA) score retrospectively. Primary endpoint was death within 30 days. The combined endpoint “major adverse event” (MAE) consisted of at least one of the primary or the secondary endpoints (EP)—need of intensive care (ITS), mechanical ventilation or dialysis. EDTA plasma samples were collected at first presentation.

### Example 2: Results

Median values of PSEP and PCT were 688 (IQR: 391-1143) pg/mL, and 1.39 (IQR: 0.385-4.29) ng/mL in the group with uncomplicated sepsis (N=66) and 1266 (IQR: 746-2267) pg/mL, p=0.0003, and 2.73 (IQR: 0.90-16.5) ng/mL in patients with septic shock or with complicated clinical course, p=0.0242, respectively. The 30-day mortality was 18.1% (n=18) overall, but in the group with septic shock 36.6% (n=15). The discrimination between survivors (n=81) and non-survivors (n=18) by ROC analysis revealed AUC values of 0.772, 0.519 and 0.802 of PSEP, PCT and qSOFA, respectively. The combination of PSEP, PCT and qSOFA by logistic regression revealed an AUC value of 0.850.
24 patients were assigned to qSOFA=0, 44 patients to qSOFA=1, 23 patients to qSOFA=2, and 8 patients to qSOFA=3.
In 62.5% of the patients with qSOFA=0 the certainty with which a complicated clinical course (CCC) is ruled-out could be improved by using additionally the algorithm PSEP <1000 pg/mL and PCT <2 ng/mL (Tab1.1).
In patients with qSOFA=1 the algorithm could differentiate between low risk and high risk of a complicated clinical course in 34.0% and 65.9%, respectively (Tab1.2).
In patients with qSOFA=2 the algorithm could indicate the majority of 73.9% to rule in a complicated clinical course and 26% to rule out (Tab1.3).
In patients with qSOFA=3 the algorithm could indicate 87.5% to rule in a complicated clinical course and 12.5% to rule out (Tab1.4).

Summary of ED Study Results:

| | | | Rule-out: | Rule-in: | | |
|---|---|---|---|---|---|---|
| | | | PSEP < | PSEP ≥ | | |
| | | | 1000 | 1000 | | |
| | | | pg/mL and | pg/mL and/ | | Non- |
| | | No | PCT < | or PCT ≥ | Survi- | survi- |
| | CCC | CCC | 2 ng/mL) | 2 ng/mL | vors | vors |
| All | 33 | 66 | | | 81 | 18 |
| n = 99 | | | | | | |
| qSOFA = 0 | 11 | 13 | 15 | 9 | 24 | 0 |
| n = 24 | | | (62.5%) | (37.5%) | | |
| qSOFA = 1 | 29 | 15 | 17 | 27 | 39 | 5 |
| n = 44 | | | (34.0%) | (65.9%) | | |
| qSOFA = 2 | 19 | 4 | 6 | 17 | 17 | 7 |
| n = 23 | | | (26%) | (73.9%) | | |
| qSOFA = 3 | 7 | 1 | 1 | 7 | 2 | 6 |
| n = 8 | | | (12.5%) | (87.5%) | | |

Calculation of the sensitivity, specificity, positive predictive value (PPV) and negative predictive value (NPV) of qSOFA, qSOFA+PCT and qSOFA+PCT+PSEP in predicting hospital mortality and septic shock:
1. Discrimination of death vs alive by receiver operating characteristics curve (ROC) analysis

| | | Sensi- | Speci- | NPV | PPV |
|---|---|---|---|---|---|
| | AUC | tivity % | ficity % | % | % |
| qSOFA + PCT + PSEP | 0.852 | 89.4 7 | 64.13 | 96.7 | 34.0 |
| qSOFA + PSEP | 0.848 | 78.95 | 73.91 | 94.4 | 39.0 |
| qSOFA + PCT | 0.824 | 84.21 | 72.83 | 95.7 | 39.0 |
| qSOFA | 0.801 | 68.42 | 77.17 | 92.2 | 38.2 |

2. Discrimination of septic shock vs uncomplicated sepsis by receiver operating characteristic curve (ROC) analysis

| | | Sensi- | Speci- | NPV | PPV |
|---|---|---|---|---|---|
| | AUC | tivity % | ficity % | % | % |
| qSOFA + PCT + PSEP | 0.817 | 73.81 | 76.81 | 82.8 | 66.0 |
| qSOFA + PSEP | 0.810 | 78.57 | 76.81 | 85.5 | 67.3 |
| qSOFA + PCT | 0.794 | 54.76 | 92.75 | 77.1 | 82.1 |
| qSOFA | 0.765 | 53.66 | 84.72 | 76.2 | 66.7 |

Sensitivity: Probability that a test result will be positive when the disease is present (true positive rate).
Specificity: Probability that a test result will be negative when the disease is not present (true negative rate).
Positive predictive value (PPV): Probability that the disease is present when the test is positive.
Negative predictive value (NPV): Probability that the disease is not present when the test is negative.

### Example 2: Individual Case Studies

Patients with qSOFA=0

Study ID 402

A 71 years old men (size 182 cm, weight 83 Kg, BMI 25) was admitted to the emergency department with fever and unclear genesis. The measured temperature was 39.7° C.

Respiratory rate, RRsyst and the GSC score were 24/min, 101 mmHg and 15, respectively, revealing a qSOFA score of 0.

PSEP and PCT were 693 pg/mL and 1.1 ng/mL indicating rule out of complicated clinical cause according to the algorithm.

The patient was admitted to the general ward and could be discharged after 12 days at home without complications.

Study ID 387

A 76 years old women was admitted to the emergency department with pneumonia (size 158 cm, weight 53 Kg, BMI 21.3). Respiratory rate, RRsyst and the GSC score were 26/min, 123 mmHg and 15, respectively, revealing a qSOFA score of 0.

PSEP and PCT were 287 pg/mL and 0.1 ng/mL indicating rule out of complicated clinical cause according to the algorithm.

The patient could be discharged at home from the emergency department.

Patients with qSOFA=1

Study ID 383

A 73 years old men (size 168 cm, weight 88 Kg, BMI=31) was admitted to the emergency department with pneumonia. Respiratory rate, RRsyst and the GSC score were 28/min, 125 mmHg and 15, respectively, revealing a qSOFA score of 1.

PESP and PCT were 3744 pg/mL and 0.56 ng/mL indicating moderate risk for prediction of complicated clinical cause and mortality risk according to the algorithm “qSOFA=1, PSEP >1000 pg/mL or PCT <2 ng/mL” indicates The PSEP concentration was >1000 pg/mL but the measured PCT value of 0.56 ng/mL was below the threshold of 2 ng/mL.

According qSOFA=1 and the very high PSEP value of 3744 pg/mL the patient was assigned to complicated clinical cause and admitted to the ICU. Instead of mechanical ventilation and dialysis the patient died after 25 days during the ICU stay.

Study ID 360

A 84 years old patient (size 162 cm, weight 60 Kg, BMI=22.9) was admitted to the emergency department with urinary tract infection. Respiratory rate, RRsyst and the GSC score were 22/min, 112 mmHg and 15, respectively, revealing a qSOFA score of 1.

PSEP and PCT values were 1517 pg/mL and 0.54 ng/mL indicating moderate risk of complicated clinical cause.

The patient was admitted to the general ward for antibiotic therapy and could be discharged after 7 days at home.

Study ID 313

A 42 years old men was admitted to the emergency department with pneumonia (size 178 cm, weight 101 Kg, BMI 31.9). Respiratory rate, RRsyst and the GSC score were 22/min, 130 mmHg and 15, respectively, revealing a qSOFA score of 1.

PSEP and PCT were 799 pg/mL and 0.26 ng/mL indicating low risk of complicated clinical cause according to the algorithm.

The patient was admitted to the general ward and received antibiotic therapy for 14 days until he was discharged at home without complications.

Study ID 458

A 87 years old men was admitted to the emergency department with urosepsis (size 160 cm, weight 70 Kg, BMI 27.3). Respiratory rate, RRsyst and the GSC score were 20/min, 147 mmHg and 11, respectively, revealing a qSOFA score of 1.

PSEP and PCT were 342 pg/mL and 6.4 ng/mL indicating high risk of complicated clinical cause according to the elevated PCT concentration. Also clinically the patient was assigned to severe sepsis underlined by a high CRP concentration of 102 mg/L.

The patient was admitted to the intensive care unit for two days and received early goal directed therapy. After further treatment at the general ward for 9 days the patient could be discharged at home without complications.

Study ID 461

A 68 years old women was admitted to the emergency department with pneumonia (size 174 cm, weight 103 Kg, BMI 34.2). Respiratory rate, RRsyst and the GSC score were 20/min, 110 mmHg and 14, respectively, revealing a qSOFA score of 1.

PSEP and PCT were 236 pg/mL and 8.1 ng/mL indicating high risk of complicated clinical cause according to the elevated PCT concentration. Also clinically the patient was assigned to severe sepsis underlined by a high CRP concentration of 102 mg/L.

The patient was admitted to the intensive care unit for three days and received early goal directed therapy. After further treatment at the general ward 7 the patient could be discharged at home without complication.

Patients with qSOFA=2

Study ID 403

A 87 years old men was admitted to the emergency department with urosepsis (size 175 cm, weight 65 Kg, BMI 21.2). Respiratory rate, RRsyst and the GSC score were 24/min, 74 mmHg and 15, respectively, revealing a qSOFA score of 2.

The algorithm “qSOFA=2, PSEP >1000 pg/mL or PCT >2 ng/mL” indicates prediction of complicated clinical cause and high mortality risk. The patient was admitted to the ICU for early goal directed therapy according to PSEP and PCT concentration of 1979 pg/mL and 16 ng/mL, respectively, measured at presentation.

After discharge from the ICU to the general ward the antibiotic therapy was continued but the patient died 5 days later.

Study ID 390

A 64 years female with abdominal pain due to acute cholecystitis was admitted to the emergency department (size 163 cm, weight 106 Kg, BMI 40). The qSOFA score at presentation was 2 (Respiratory rate, RRsyst and the GSC score were 28/min, 108 mmHg and 3).

PSEP and PCT values were 1858 pg/mL and 292 ng/mL indicating underlying sepsis with complicated clinical cause.

The patient was admitted to the ICU for early goal directed therapy and needed mechanical ventilation. After 5 days on the ICU and 10 days on the general ward the patient could be discharged at home.

Study ID 357

A 79 years old female (size 160 cm, weight 70 Kg, BMI 27) was admitted to the emergency department with urosepsis. Respiratory rate, RRsyst and the GSC score were 25 min, 140 mmHg and 13, respectively, revealing a qSOFA score of 2.

PESP and PCT were 1810 ng/L and 2.15 μg/L indicating high risk for prediction of complicated clinical cause and mortality risk according to the algorithm “qSOFA=1, PSEP >1000 pg/mL, PCT >2 ng/mL”.

The patient received early goal directed therapy and antibiotic therapy for 18 days at the general ward until discharge at home.

Patients qSOFA=3

Study ID 374

A 87 years old men (size 170 cm, weight 65 Kg, BMI 22.5) suffered from urinary tract infection and was admitted to the emergency department. Respiratory rate, RRsyst and the GSC score were 24/min, 80 mmHg and 3, respectively, revealing a qSOFA score of 3.

The algorithm “qSOFA=3, PSEP >1000 pg/mL or PCT >2 ng/mL” indicated prediction of complicated clinical cause and high mortality risk. The PSEP concentration of 8238 pg/mL was extremely high whereas and the PCT value was below 2 ng/mL (0.91 μg/L). Although PCT was <2 ng/mL high risk of worse outcome and mortality risk of >50% could be expected.

The high PSEP concentration of 8238 pg/mL might also be influenced through acute kidney disease (AKD) which was indicated by a measured creatinine concentration of 1022 μmon. AKD occurs commonly in sepsis and contributes to mortality risk significantly.

The patient was admitted to the intensive care unit for early goal directed therapy and died after 5 days.

Study ID 373

A 82 years old men with pneumonia was admitted to the emergency department. Respiratory rate, RRsyst and the GSC score were 24/min, 100 mmHg and 7, respectively, revealing a qSOFA score of 3.

The algorithm “qSOFA=3, PSEP >1000 pg/mL or PCT >2 ng/mL” indicates prediction of complicated clinical cause and high mortality risk. The PSEP concentration was >1000 pg/mL (1407 ng/L) but the measured PCT value of 0.37 ng/mL was below the threshold of 2 ng/mL of the algorithm and below 0.5 ng/mL.

According qSOFA=3 and PSEP >1000 pg/mL the patient was assigned to complicated clinical cause and admitted to the ICU. Despite intensive care with mechanical ventilation and dialysis during the ICU stay the patient died after 7 days.

Study ID 381

A 78 years old female was admitted to the emergency department urinary tract infection. Respiratory rate, RRsyst and the GSC score were 30/min, 91 mmHg and 12 revealing a qSOFA score of 3.

The PSEP concentration of 504 pg/mL was below the threshold of 1000 pg/mL whereas the PCT value was 2 pg/ml. According to the algorithm qSOFA=3, PSEP <1000 pg/mL but PCT ≥2 ng/mL complicated clinical causes like severe sepsis or septic shock could not be excluded. The patient was admitted to the general ward for antibiotic treatment and was discharged after 9 days without complications.

Study ID 389

A 75 years old men was admitted to the emergency department with urosepsis (size 165 cm, weight 100 Kg, BMI 36.7). Respiratory rate, RRsyst and the GSC score were 24/min, 100 mmHg and 11, respectively, revealing a qSOFA score of 3.

The PSEP concentration of 3496 pg/mL was above the threshold of 1000 pg/mL and the PCT value was 25.6 ng/mL. According to the algorithm qSOFA=3, PSEP >1000 pg/mL and PCT ≥2 ng/mL complicated clinical causes like severe sepsis or septic shock could not be excluded.

The patient was admitted to the ICU for 4 days because of need of dialysis due to acute kidney disease. After discharge to the general ward the patient died after 9 days during hospital stay.

### CONCLUSIONS

In patients with qSOFA=0 or qSOFA=1 and a PCT concentration of <2 ng/mL and Presepsin concentration of <1000 pg/mL, a clinical course without risk of complications may be assumed for the patient. If one of the two biomarkers exceeds the respective limit value, a clinical course without risk of complications cannot be safely eliminated. In a study of 99 patients with suspected sepsis in the emergency room, for 24 patients with qSOFA=0 and for 44 patients with qSOFA=1, a complicated clinical course only could be excluded in 62.5% and 65.9% of cases respectively, although these patients exhibited a “negative” qSOFA (<2)

In patients with qSOFA=2 and a PCT concentration of <2 ng/mL and a Presepsin concentration of <1000 pg/mL, a good prognostic course can be expected, despite the “positive” qSOFA score. In the above study 26% of patients with qSOFA=2 had an uncomplicated clinical course. On the other hand, patients with a PCT concentration of >2 ng/mL and/or a Presepsin concentration of >1000 pg/mL are at an increased risk of a complicated clinical course. Patients like this must be monitored by medical staff, if necessary in the intensive care unit. In the same emergency study in 23 patients with qSOFA=2, a complicated clinical course was reliably predicted in 73% of cases.

In patients with qSOFA=3 and a PCT concentration and Presepsin concentration of over 2 ng/mL and ≥1000 pg/mL respectively, severe bacterial sepsis or septic shock is ensured with high mortality.

Thus, the findings of the present invention show that the combination of qSOFA with Presepsin and Procalcitonin is more accurate in predicting a complicated clinical course of sepsis and hospital mortality than the qSOFA score alone. Accordingly, the combined assessment of qSOFA, Presepsin and Procalcitonin according to the proposed algorithm improves the risk stratification of patients with suspected sepsis admitted to the emergency department significantly.

1. A method for aiding in the risk assessment of a patient with suspected sepsis, comprising
(a) determining the amount of the biomarker Presepsin in a sample from a patient with suspected sepsis who has a known qSOFA (quick Sequential Organ Failure-Assessment) score of 0, 1, 2 or 3,
(b) determining the amount of the biomarker Procalcitonin (PCT) in a sample from the patient,
(c) comparing the amounts determined in steps (b) and (c) to reference amounts, and
(d) aiding in the risk assessment of a patient with suspected sepsis.

2. The method of claim 1, wherein the patient is a human patient.

3. The method of claim 1, wherein the sample is a blood, serum or plasma sample.

4. The method of claim 1, wherein the patient is a patient who presents with suspected sepsis.

5. The method of claim 1, wherein the risk of mortality, such as in-hospital mortality, a complicated clinical course, severe sepsis and/or septic shock is assessed.

6. The method of claim 5, wherein the complicated clinical course is defined as the need for organ support measures required during intensive care unit (ICU) stay, such as administration of intravenous fluids, vasopressors, mechanical ventilation or renal replacement therapy.

7. The method of claim 1, wherein the subject has a known qSOFA (quick Sequential Organ Failure-Assessment) score of 0 or 1.

8. The method of claim 7, wherein an amount of Presepsin in the sample from the subject which is larger than the reference amount for Presepsin and/or an amount of PCT in the sample from the subject which is larger than the reference amount for PCT is indicative for a subject who is at risk.

9. The method of claim 7, wherein an amount of Presepsin in the sample from the subject which is lower than the reference amount for Presepsin and an amount of PCT in the sample from the subject which is lower than the reference amount for PCT is indicative for a subject who is not at risk.

10. The method of claim 1, wherein the subject has a known qSOFA (quick Sequential Organ Failure-Assessment) score of 2 or 3.

11. The method of claim 10, wherein an amount of Presepsin in the sample from the subject which is larger than the reference amount for Presepsin and/or an amount of PCT in the sample from the subject which is larger than the reference amount for PCT is indicative for a subject who is at risk.

12. The method of claim 10, wherein an amount of Presepsin in the sample from the subject which is lower than the reference amount for Presepsin and an amount of PCT in the sample from the subject which is lower than the reference amount for PCT is indicative for a subject who is not at risk.

13. The method of claim 1, further comprising recommending or initiating a suitable therapeutic measure.

14. The method of claim 13, wherein the therapeutic measure is selected from recommended guidelines for management of sepsis, if the subject has been assessed to be at risk.

15. The method of claim 13, wherein the therapeutic measure may be treatment of infection or further investigation or other aspects of care deemed necessary by a practitioner, if the subject has been assessed to be not at risk.

16. The method of claim 1, wherein the reference amount for Presepsin is within a range from about 500 pg/mL to about 1500 pg/mL or about 750 pg/mL to about 1250 pg/mL, and/or wherein the reference amount for PCT is within a range from about 1.5 ng/mL to about 2.5 ng/mL.

17. A method for aiding in the risk assessment of a patient with suspected sepsis, comprising
(a) obtaining the patient's qSOFA (quick Sequential Organ Failure-Assessment) score,
(b) determining the amount of the biomarker Presepsin in a sample from the patient with suspected sepsis,
(c) determining the amount of the biomarker Procalcitonin (PCT) in a sample from the patient,
(d) comparing the amounts determined in steps (b) and (c) to reference amounts, and
(e) aiding in the risk assessment of the patient.

18. The method of claim 17, wherein the patient's qSOFA score is based on the patient's respiratory rate (>22/min), the patient's systolic blood pressure (<100 mmHg), and the presence or absence of an altered mentation (GCS <15).

19. A computer-implemented method for the assessment of a patient with suspected sepsis, comprising
(a) receiving at a processing unit
(a1) a value for the amount of the biomarker Presepsin in a sample from a patient with suspected sepsis who has a known qSOFA (quick Sequential Organ Failure-Assessment) score of 0, 1, 2 or 3 and
(a2) a value for the amount of the biomarker Procalcitonin in a sample from the patient,
(b) processing the values received in step (a) with the processing unit, wherein said processing comprises
(b1) retrieving from a memory one or more threshold values for the amount of the biomarker Presepsin, and one or more threshold values for the amount of the biomarker Procalcitonin,
(b2) comparing the values received in step (a) with the respective threshold values retrieved in step (b1), and
(c) providing an assessment of the patient via an output device, wherein said assessment is based on the results of step b).

20. A computer-implemented method for the assessment of a patient with suspected sepsis, comprising
(a) receiving at a processing unit
(a1) a value for the patient's qSOFA (quick Sequential Organ Failure-Assessment) score,
(a2) a value for the amount of the biomarker Presepsin in a sample from the patient, and
(a3) a value for the amount of the biomarker Procalcitonin in a sample from the patient,
(b) processing the values received in step (a) with the processing unit, wherein said processing comprises
(b1) retrieving from a memory a threshold value for the qSOFA score, one or more threshold values for the amount of the biomarker Presepsin, and one or more threshold values for the amount of the biomarker Procalcitonin,
(b2) comparing the values received in step (a) with the respective threshold values retrieved in step (b1), and
(c) providing an assessment of the patient via an output device, wherein said assessment is based on the results of step b).

21. The method of claim 19, wherein the output device is a display, configured for presenting the assessment.

22. The method of claim 21, wherein the value for the amount of the biomarker Presepsin and/or Procalcitonin is the value for the amount of the biomarker in a blood, serum or plasma sample.
